(19) United States
(12) Patent Application Publication
(10) Pub. No.: US 2003/0149461 A1
(43) Pub. Date: Aug. 7, 2003

(54) Re-usable re-freezable cooler and re-usable re-freezable/re-heatable therapy/hot/cold wraps

(76) Inventor: Tracy J. Johnson, Lake Oswego, OR (US)

Correspondence Address: DELLETT AND WALTERS, 310 S.W. FOURTH AVENUE, SUITE 1101, PORTLAND, OR, 97204, US

(21) Appl. No.: 10/319,858

(22) Filed: Dec. 12, 2002

Related U.S. Application Data
(63) Continuation-in-part of application No. 10/237,137, filed on Sep. 6, 2002, now abandoned.
(60) Provisional application No. 60/343,113, filed on Dec. 22, 2001.
(60) Provisional application No. 60/330,299, filed on Oct. 17, 2001.
(60) Provisional application No. 60/317,846, filed on Sep. 6, 2001.

(51) Int. Cl. A61F 7/00

(52) U.S. Cl. 607/108; 607/112; 607/114

(57) ABSTRACT

A flexible, foldable re-freezable cooler is adapted to fold relatively flat for storage or freezing, and to expand to a use configuration for receiving items to be cooled or kept cool therein. A therapy wrap, for human or animal use, is adapted for easy cold therapy application.

### BACKGROUND OF THE INVENTION

This invention relates to cooling, and more specifically, to portable re-freezable coolers for cooling food, beverages, limbs, and the like and adapted for freezing and storing in a freezer prior to use. Further, the invention relates to therapy wraps for use in injury prevention and therapy.

The use of coolers for items is well known. Such coolers may comprise, for example, relatively rigid containers for holding food and beverage, wherein ice or reusable packaged refrigerant elements are placed in the container along with the food or beverage. However, in the case of ice, as the ice melts, a volume of liquid pools in the bottom of the container, which might be absorbed by the food in the container, and which eventually must be drained. Also, more ice must be bought or made to replace the melted ice, or to refill the container when it is used again after a period of non-use. The rigid cooler takes up a given amount of space, whether empty or full, and presents storage problems, and can be in the way, especially in the case of use in a small camper or boat, for example. Cleaning such rigid coolers after use can be awkward, since the bulky cooler is unlikely to fit into a typical kitchen sink for cleaning.

In the case of using reusable packaged refrigerant elements or re-freezable cooling blocks, typically a freezable material such as a gel is contained inside a flexible bag or rigid block shape, wherein the block or bag is stored in the freezer prior to use to cool the element below freezing, and then is placed inside the cooler container at the time of use. Having to separately freeze and store the cooling blocks and then remove them from the freezer to place them in the cooler can be somewhat of an inconvenience.

U.S. Pat. No. 5,035,122, illustrates a rigid cooler container with cooling gel in the walls thereof. To use this device, the entire container is placed in a freezer in an open condition. Then, when it is time to use the cooler, it may be taken from the freezer and receive food or beverages or the like therein. However, the cooler can take up a good deal of space in the freezer, which is inconvenient. Also, given its rigidity, it can be awkward to fit it into a freezer, which is likely to have other items taking up a good deal of space that must be re-arranged to accommodate the cooler for re-freezing. Also, the rigid walls of the cooler typically result in unusable space within the cooler, as items which would normally be put inside the cooler are likely to have irregular shapes or shapes that do not conform to the generally straight or rectangular walls, resulting in inefficient packing. Over time, after multiple freezing/thaw cycles, the rigid plastic walls of the cooler may have a tendency to become brittle and fail.

Localized cooling is often used as a therapy for pain and injuries arising from exercise or overuse of a joint, etc., in both humans and animals. Typically, ice is applied to an area on or around the injury. However, as the ice melts, the resultant water can be messy. Also, positioning and holding the ice against the treatment area is somewhat of a problem and frostbite can result. U.S. Pat. No. 5,215,080 shows an attachment device for holding ice therein and against an injury site, while keeping the water from the melting ice contained. But, with this device, one must still prepare and insert ice cubes into the device.

In the case of animals, for example, horses and especially in racing horses, use of cold therapy can reduce the chance of injury to the animal, and can minimize recovery time after a race. However, it is difficult to hold ice packs and the like on a horse's legs, and unless a trainer or other person remains with the horse holding the ice on the affected area, it is likely that the horse will scrape or knock the ice pack off.

Further, with therapy wraps in general, whether for humans or animals, it is desirable to be able to apply cooling (or heating) directly to a specific site.

### SUMMARY OF THE INVENTION

In accordance with the invention, a collapsible cooler carries plural freezable compartments therewithin. Before use, the container may be stored in a freezer, in a collapsed state, minimizing the space required in the freezer. When it is time to use the cooler, it may be removed from the freezer and restored to an un-collapsed state.

Accordingly, it is an object of the present invention to provide an improved cooler.

It is a further object of the present invention to provide an improved collapsible cooler.

It is yet another object of the present invention to provide an improved cooler that may be collapsed to a minimal size for storage and freezing.

It is another object of the present invention to provide an improved re-freezable cooler that folds flat for storage or for freezing.

It is still another object of the present invention to provide an improved cooler that is flexible.

It is another object of the present invention to provide an improved cooler that is easily cleaned.

It is a further object of the present invention to provide an improved cold wrap for use on humans and animals.

It is yet another object of the invention to provide an improved cold therapy wrap for use on horses and like animals.

It is a further object of the invention to provide improved therapy wraps for heat or cold application.

The subject matter of the present invention is particularly pointed out and distinctly claimed in the concluding portion of this specification. However, both the organization and method of operation, together with further advantages and objects thereof, may best be understood by reference to the following description taken in connection with accompanying drawings wherein like reference characters refer to like elements.

### BRIEF DESCRIPTION OF THE DRAWINGS

FIG. 1 is a perspective view of a collapsible re-useable re-freezable cooler in accordance with the invention, wherein the container is closed and is in its expanded form;

FIG. 2 is a side view of the container of cooler of FIG. 1, illustrating the opening thereof;

FIG. 3 is a rear view of the cooler of FIG. 1;

FIG. 4 is a side view of the cooler of FIG. 1, wherein the cooler is in a collapsed state;

FIG. 5 is a sectional view of the beverage container cooler taken along line 11-11 of FIG. 2;

FIG. 6 is a partial cut away view of a therapy wrap according to another embodiment of the invention;

FIG. 7 is a view of a strap for use with the therapy wrap of FIG. 6;

FIG. 8 is a perspective view of the therapy wrap of FIG. 6 as worn by a user;

FIG. 9 is a perspective view of the use of the therapy wrap with the strap;

FIG. 10 is a perspective view of a therapy wrap according to the invention for use with a horse or other animal;

FIG. 11 is a view of the horse therapy wrap of FIG. 10 in an unwrapped configuration;

FIG. 12 is an end view of the horse therapy wrap;

FIG. 13 is a photographic view of a back therapy wrap;

FIG. 14 is a cut away view of the back therapy wrap of FIG. 13, illustrating the placement of the cooling/heating elements therein;

FIG. 15 is a photographic view of a wrist/carpal tunnel therapy wrap;

FIG. 16 is a cut away view of the wrist/carpal tunnel therapy wrap of FIG. 15, illustrating the placement of the cooling/heating elements therein;

FIG. 17 is a photographic view of an elbow therapy wrap;

FIG. 18 is a cut away view of the elbow therapy wrap of FIG. 17, illustrating the placement of the cooling/heating elements therein;

FIG. 19 is a photographic view of a knee therapy wrap;

FIG. 20 is a cut away view of the knee therapy wrap of FIG. 19, illustrating the placement of the cooling/heating elements therein;

FIG. 21 is a photographic view of a shoulder therapy wrap;

FIG. 22 is a cut away view of the shoulder therapy wrap of FIG. 21, illustrating the placement of the cooling/heating elements therein;

FIG. 23 is a photographic view of a face therapy mask;

FIG. 24 is a cut away view of the face therapy mask of FIG. 23, illustrating the placement of the cooling/heating elements therein;

FIG. 25 is a front view drawing of a shoulder therapy wrap illustrating placement of cooling/heating elements therein in phantom;

FIG. 26 is a rear view thereof;

FIG. 27 is a perspective view thereof as worn on a person;

FIG. 28 is a view of the face therapy mask of FIG. 23, illustrating the placement of the cooling/heating elements therein in phantom;

FIG. 29 is a front view of an alternate wrist therapy wrap;

FIG. 30 is a rear view of the wrist therapy wrap of FIG. 29, illustrating the placement of the cooling/heating elements therein in phantom;

FIG. 31 is a view of the wrist therapy wrap of FIG. 29, showing placement when worn by a user.

FIG. 32 is a view of a knee therapy wrap illustrating the placement of the cooling/heating elements therein in phantom;

FIG. 33 is a view of a modified version of the knee therapy wrap of FIG. 32, showing placement when worn by a user;

FIG. 34 is a view of an elbow therapy wrap illustrating the placement of the cooling/heating elements therein in phantom;

FIG. 35 is a view of a horse fetlock therapy wrap illustrating the placement of the cooling/heating elements therein in phantom;

FIG. 36 is a side view of the wrap of FIG. 35;

FIG. 37 is a view of the horse fetlock therapy wrap when installed on a horse;

FIG. 38 is a view of an ankle therapy wrap as worn by a user;

FIG. 39 is a view of a lower back therapy wrap illustrating the placement of the cooling/heating elements therein in phantom;

FIG. 40 is a view of placement of the lower back therapy wrap of FIG. 39 when worn by a user;

FIG. 41 is a view of a universal therapy wrap, illustrating the placement of the cooling/heating elements therein in phantom; and

FIG. 42 is a view of a beer keg cooler wrap as installed

### DETAILED DESCRIPTION

The system according to a preferred embodiment of the present invention comprises a portable collapsible re-useable re-freezable cooler and therapy wraps.

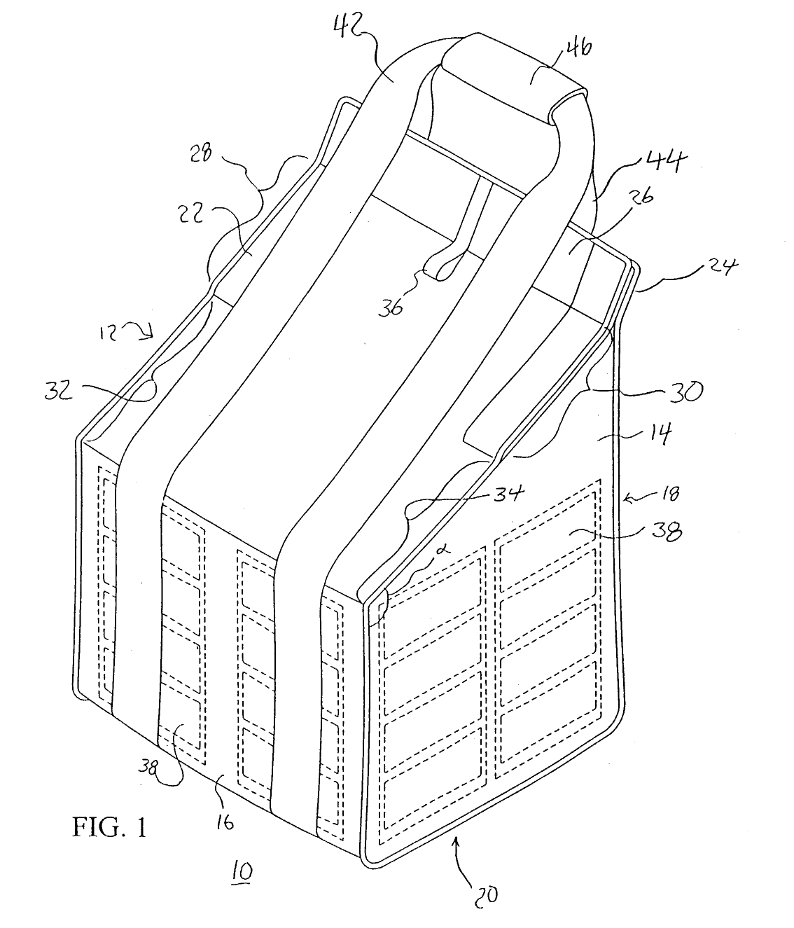

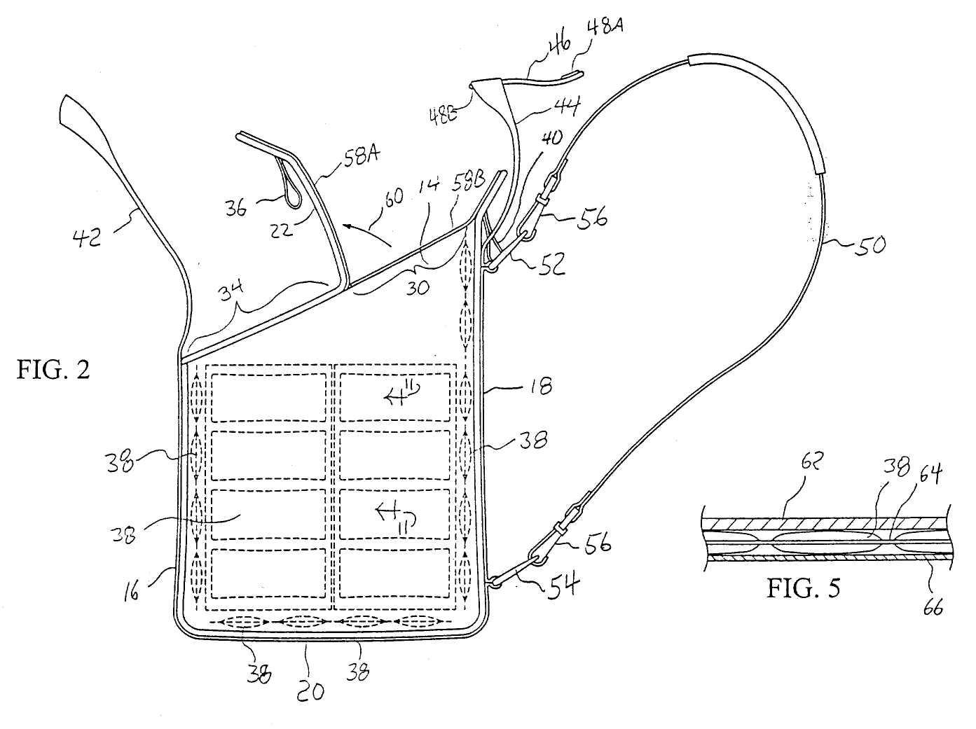

Referring to FIG. 1, a perspective view of a collapsible re-useable re-freezable cooler in accordance with the invention, wherein the container is closed and is in its expanded form, the cooler 10 is suitably of a generally rectangular foot print, having left and right side walls 12, 14, front and back walls 16, 18, bottom wall 20 and top wall 22. While the bottom ends of the side walls are generally straight, the upper ends angle upwardly when considered from the front to back of the cooler at an angle α, which is approximately 30° in the preferred embodiment. As such, top portion 22 angles upwardly. The upper end of back portion 18 and top portion 22 define laterally extending tab portions 24, 26, suitably extending the entire width of the cooler. Along the interior faces of these tab portions, mating hook and loop type fasteners are provided to enable the tab portions of the top and back to be removably secured together. The top portion and the left and right side portions also carry corresponding hook and loop fastener portions on inside faces in the regions 28, 30, for removably securing the lateral sides of the top portion to the sides 12, 14, in the areas of regions 28, 30. The remainder of the top portion edges in the regions 32, 34, extending from the lower side edges of the top portion to a position mid way along the length of the top portion, are secured to the mating side portion edges. A loop member 36 is attached to the front face of the top portion, centrally positioned at the upper edge of the top. A corresponding loop member 40 (visible in FIG. 2) may be provided on the back face of the cooler.

Still referring to FIG. 1, together with FIG. 2, which is a side view of the container of the cooler of FIG. 1, illustrating the container in an open state, it may be observed that, illustrated in phantom, plural cooling elements 38 are positioned along the inner faces of the left, right, front, back, and bottom sides of the cooler. A handle system is provided by front handle strap 42 and rear handle strap 44 which suitably extend up the front and rear faces of the cooler, forming a loop configuration. The handles a preferably secured to the cooler near the top of the front and rear faces, but are otherwise free from securement to the cooler along the top face and in a short region near the top of the rear face of the cooler. A handle wrap member 46 is suitably secured to the rear handle 44, and includes mating hook and loop portions 48A, 48B thereon, whereby the handle wrap may be selectively wrapped and secured together to maintain the front and rear handle portions together as in FIG. 1, or may be unwrapped to allow the front and rear handle portions to be separate as in FIG. 2.

Left and right backpacking type strap members 50 (shown in FIG. 2 only) are also detachably provided, connecting to corresponding upper and lower loops 52, 54, at the rear face of the cooler via clip members 56.

Note that while the embodiment illustrated herein employs freezable cooling elements 38 on the left and right sides of the cooler, a suitably effective cooler is also provided employing no such cooling elements in the sides, instead using the elements only along the front, bottom and back faces of the cooler.

In use, when the cooler is in the closed configuration of FIG. 1, it may be opened by the user by first separating the hook and loop fastener 48 in the handle wrap member 46, thereby separating the front and rear carrying handle portions. Now, the cooler may be opened by pulling the upper portions of the top and back faces apart, pulling on the loop members 36 and 40 to assist in the separation, if necessary. Corresponding hook and loop fasteners 58 (comprising portions 58A and 58B) are positioned along the inner peripheral faces of the top, side and upper edge of the back faces of the cooler, and allow the separation of a portion of the upper face of the cooler in the direction of arrow 60, enabling access to the interior of the cooler, to add or remove items therefrom. To re-close the cooler, the user simply moves the top face back in the reverse direction of arrow 60, and presses the hook and loop fasteners 58 together. Then, if desired, the carrying handle strap 42, 44 can be brought together at the upper ends thereof, and joined together by wrapping handle wrap member 46 around the two handle portions and joining the hook and loop fasteners 48A, 48B together. The handle wrap 46 provides a cushioned carrying handle.

While in the illustrated embodiment, the top wall opens only part way down as a result of the configuration of the hook and loop fasteners. The lower peripheral side portions of the top wall are suitably secured (e.g., by sewing or other means) to the side walls. This provides for easier re-sealing of the top via the hook and loop fasteners, since the flexible nature of the material from which the cooler is constructed can make re-sealing of the hook and loop fasteners more of a two handed operation if the top opens completely along the extent of the side walls. That said, other embodiments employ a top that does open completely, but the partial opening configuration can be more desirable in a larger size cooler if one handed closing is an issue.

FIG. 5, which is a sectional view of the cooler taken along line 11-11 of FIG. 1, illustrates the construction of the walls thereof that carry cooling elements 38 therein. The exemplary wall of the cooler, in this case, a portion of side wall 14 is shown, comprises as outer shell 62, a central “ice blanket” 64 which comprises plural cooling elements 38, and an inner layer 66. In a preferred embodiment, the outer shell is an insulated neoprene, while the inner layer comprises a rubber impregnated linen or a rubber impregnated nylon fabric, which is stretchable. The ice blanket 64 comprises multiple “pillow shaped” cooling elements 38, which may be joined together via the ice blanket in groups of one or more such elements 38. Those portions of the cooler walls that do not include the cooling elements, such as the top portion 22, suitably comprise the neoprene layer only.

The material inside the cooling elements is suitably a polyethylene glycol encased in a flexible plastic, such as Cryopak “Flexible Ice Blankets” by Cryopak Corporation of Vancouver, British Columbia. A suitable cooling element construction is described in U.S. Pat. No. 4,931,333, the disclosure of said patent being herein incorporated by reference. The size, spacing and configuration of the cooling elements may be varied to provide different flexibility characteristics. For example, a larger number of smaller size elements may be used, which would provide a somewhat more flexible wall construction when the elements were in a frozen, solid state.

The construction of the walls of the cooler having the ice blanket therein, with the use of rubber impregnated linen or rubber impregnated nylon fabric for the inner layer, the neoprene outer layer, and the ice blanket in the middle (which, is suitably relatively free to move or “float” somewhat between the two layers, as constrained by the outer edges of the cooler, enables the walls of the cooler to stretch. The ice blanket is suitably flexible, being made of a plastic material, and able to flex in the webbing portions thereof between the cooling elements 38, even when the cooling elements are in a solid state as a result of freezing.

The rubber impregnated inner layer is further advantageous in that it is not porous or absorbent, and therefore will resist against moisture or condensation on the cooling elements 38 from passing therethrough.

Referring now to FIG. 4, which is a side view illustrating the cooler is in a collapsed state, the construction and configuration of the cooler enables it to be collapsed when empty, whereupon it takes up a relatively small overall volume. The cooler, in this collapsed configuration, can be put into a freezer compartment to freeze the cooling elements 38. The collapsed configuration minimizes the volume occupied by the cooler in the freezer, which makes for easier use. Then, when it is desired to use the cooler, it is simply removed from the freezer compartment, and items may be placed inside, such as food or beverage, for example. The cooling elements 38 together with the insulative properties of the neoprene walls will provide hours of cool storage even though the ambient temperature may be well above desired storage temperatures for the food/beverage.

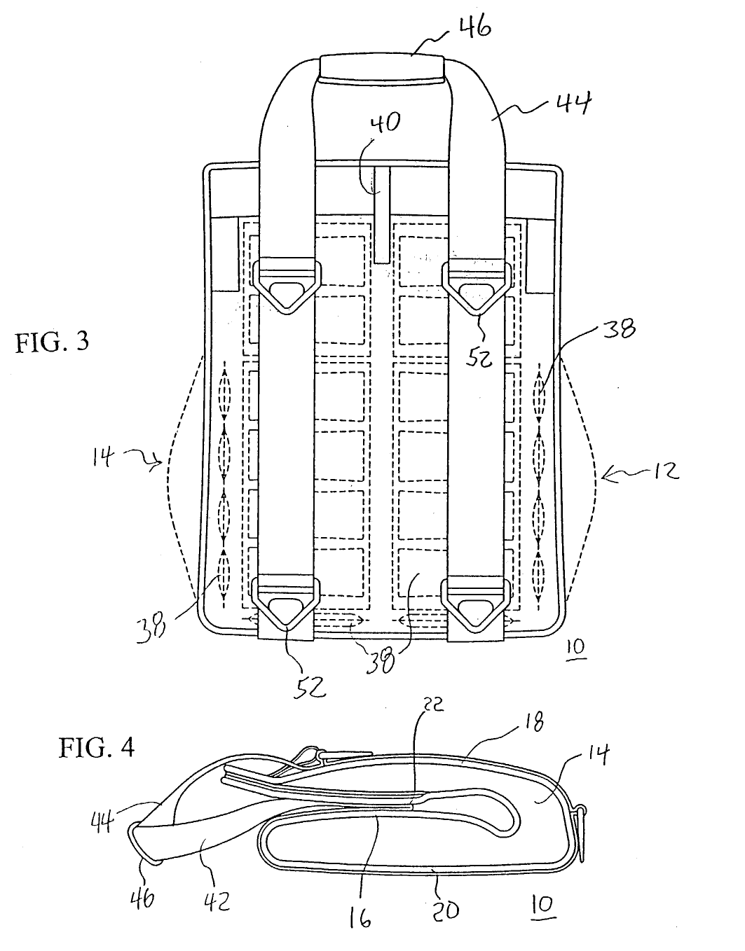

Referring to FIG. 3, a rear view of the cooler 10, the flexible/stretchable nature of the materials and construction used in the side walls enables the maximum use of cooler space. In FIG. 3, the side walls are illustrated in phantom as bulging outwardly as a result of the interior of the cooler being stuffed with a large number of items. This stretching ability provides an advantage over rigid hard sided coolers, since irregular shaped items or more items can be jammed into the cooler of the present invention than would be accommodated by a prior art rigid cooler of cooler of similar basic dimensions.

Use of the removable backpack type straps enable the cooler to be worn as a backpack if desired. Or, it may be carried by use of the carrying handles and the handle wrap member. Further embodiments of the cooler include interior and exterior pockets, which can be provide with closures or which can be open type pockets, for additional storage capabilities.

The materials and construction of the cooler enable easy cleaning, whether by hand or by machine washing in, for example, a dishwasher.

Thus, in accordance with the illustrated embodiment of the invention an improved flexible re-freezable cooler is provided. The cooler may be folded flat to store or to freeze, minimizing the amount of space required for storage and for freezing. The walls of the cooler are flexible, and enable the cooler to be over stuffed with items if desired. It may be carried in purse or bag-like fashion, or, may be worn in the manner of a backpack or over the shoulder, by use of removable straps. It is easily cleaned after use, whether by hand or by machine washing.

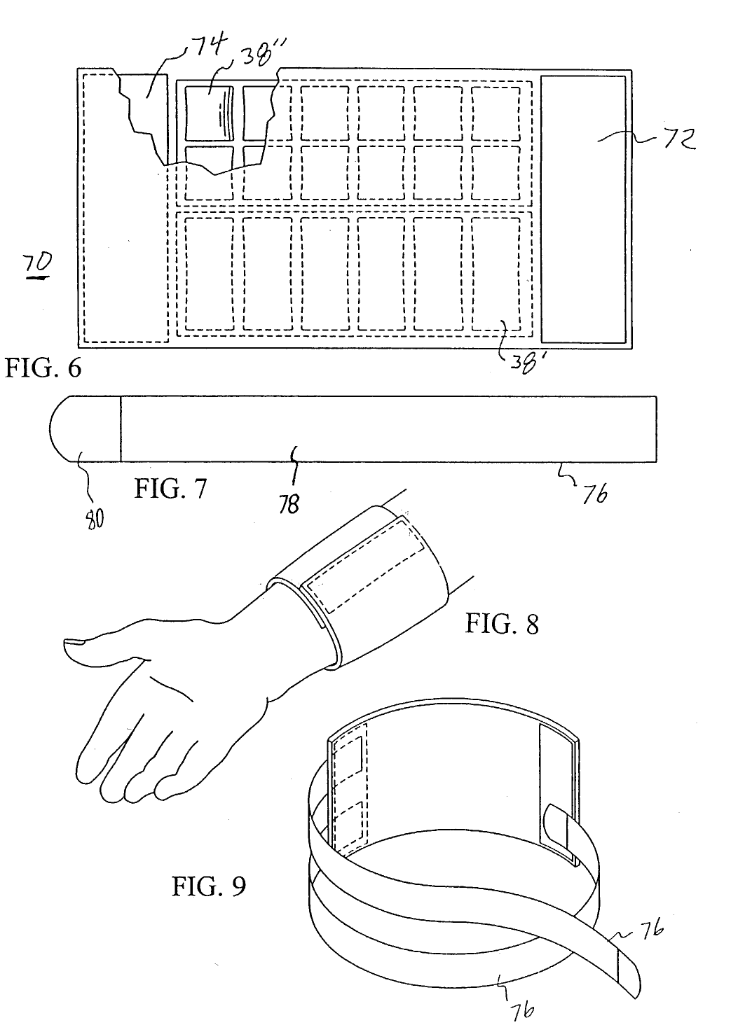

Referring to FIG. 6, which is a partial cut away view of a therapy wrap according to another embodiment of the invention, plural freezable cooling elements 38′ are employed, which may be somewhat elongate, or, as at 38″, more square-like. In a manner corresponding to the cooler described above, the inner layer of the therapy wrap comprises a rubber impregnated linen or a rubber impregnated nylon fabric, which is stretchable, while the outer shell is an insulated neoprene. The overall configuration of the wrap is somewhat longer than wide and includes corresponding hook and loop type fasteners 72 and 74 at distal ends of the wrap. Referring to FIG. 8, a perspective view of the therapy wrap of FIG. 6 as worn by a user on the wrist, the wrap in a typical use as a therapy for carpal tunnel problems, for example, is wrapped around the affected part of the body (the wrist or arm in this example) and the hook and loop fastener portions 72, 74 are engaged to hold the wrap in place.

In the case of using the wrap on other parts of the body, for example on a leg or knee, a larger diameter can be accommodated by use of a strap 76 (FIG. 7). The strap 76, is elongate and relatively narrow in the preferred embodiment and is suitably made with a loop type portion of hook and loop material thereon, along and extent of the strap. At one end 80 of the strap, a smaller hook type material portion is provided. Thus, in use, referring to FIG. 9, one or more straps 76 are removably attached to the respective portions of the hook and loop material 72, 74 on the wrap, enabling the wrap to be worn on a much larger diameter limb of the user. Thus, for example, the wrap can-be place at a knee or on a leg portion or upper arm portion or other body portion of the wearer, if desired, to hold the cold portion of the wrap against a desired portion of the body.

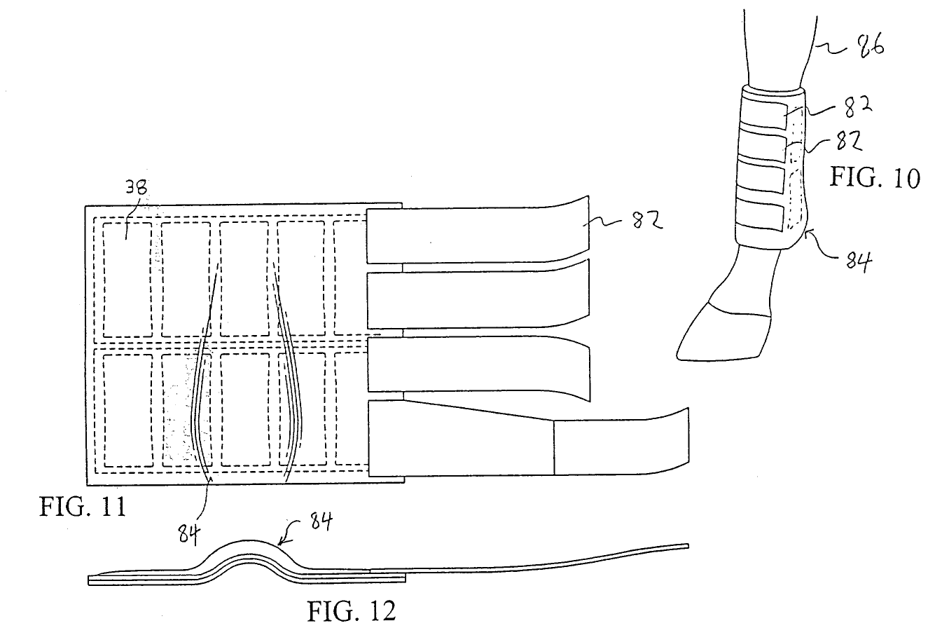

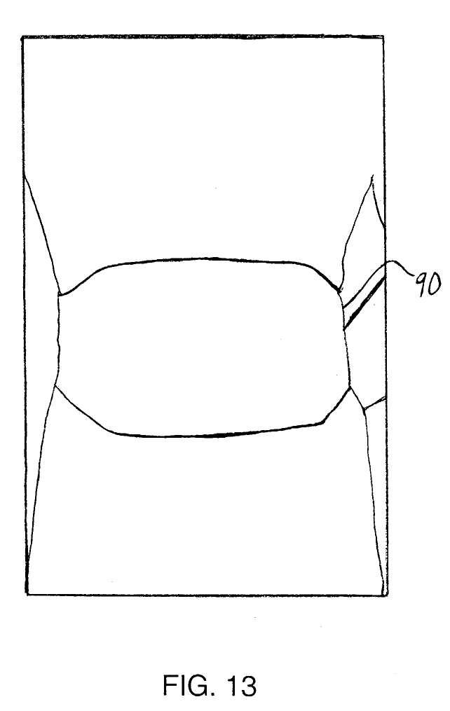

For therapy use on animals, an alternative embodiment is provided. FIG. 10 is a perspective view of a therapy wrap according to the invention for use with a horse or other animal. Referring to FIG. 10, together with FIG. 11, a view of the horse therapy wrap of FIG. 10 in an unwrapped configuration, and FIG. 12, an end view of the horse therapy wrap, the wrap includes plural, re-freezable cooling elements 38 therein, aligned so as to have their long axes in the vertical direction of FIG. 11. One or more straps 82 are attached to the wrap at one end thereof, and suitably comprise hook type material of hook and loop fasteners. The outer surface of the horse therapy wrap is of a material which will be engaged by the hook type fasteners, for example and nylon type material, or loop type fastener material.

The horse therapy wrap includes a fetlock receiving portion 84, which provide an indented or bowed out portion (relative to the inside or outside of the wrap).

Thus in use, the wrap is placed on the horse's leg 86, such that the fetlock receiving portion of the wrap is positioned over the horse's fetlock. The engaging straps are then wrapped around so as to secure the wrap on the horse's leg, by engaging the outer surface of the wrap. The vertical orientation and spacing of the cooling elements enables the wrap to conform to the shape of the horse's leg, even when the cooling elements are frozen and relatively inflexible.

While illustrated for use with a horse, other animals may be accommodated, with modification of the fetlock receiving portion or the like so as to fit the configuration of the particular animal for which the therapy wrap is intended.

While the above use discusses the wrap as a cold wrap, by heating the wrap, a heat therapy treatment may also be accomplished.

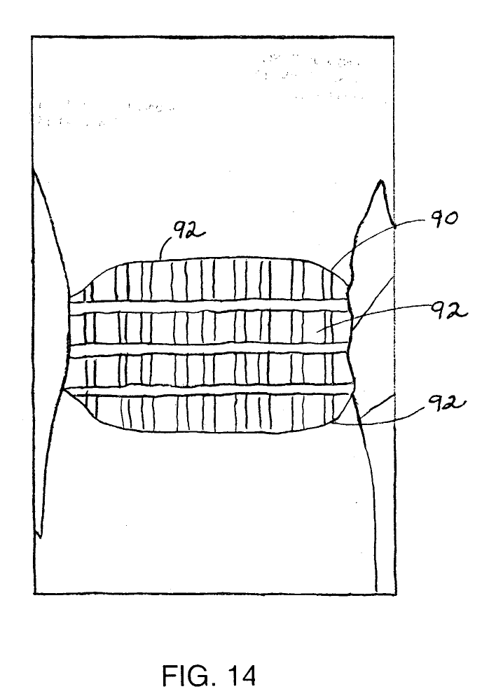

Various wrap configurations are encompassed by embodiments of the invention, as illustrated in FIGS. 13-22. FIG. 13 shows a back therapy wrap 90, worn on the lower back of a person. FIG. 14 is a cut away view of the back therapy wrap of FIG. 13, illustrating the placement of the cooling/heating elements 92 therein. The cooling/heating elements correspond to the cooling elements as discussed in connection with the other embodiments hereinabove.

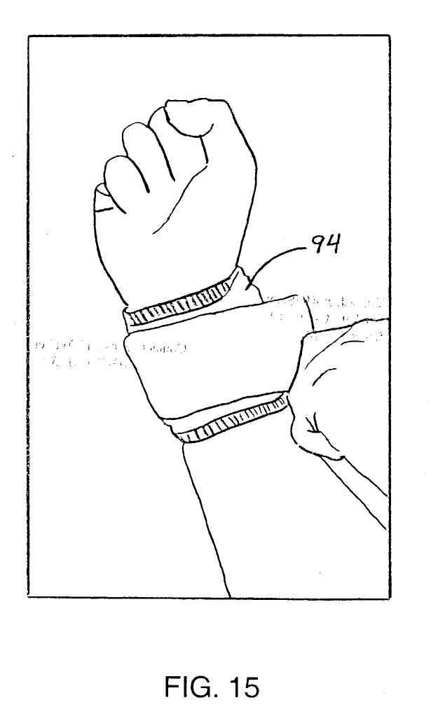

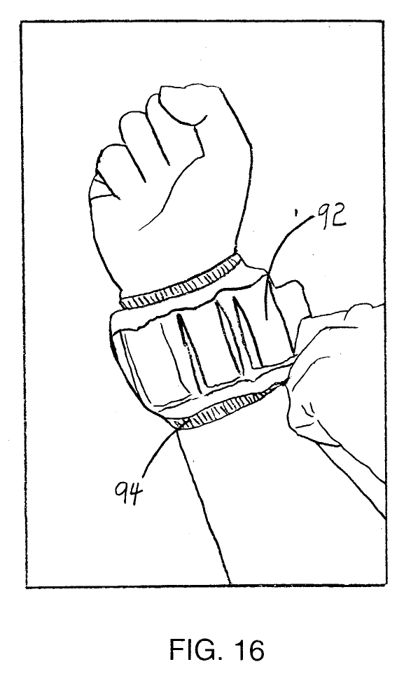

FIG. 15 is a photographic view of a wrist/carpal tunnel therapy wrap 94, while FIG. 16 is a cut away view thereof, illustrating the placement of the cooling/heating elements 92 therein. The elements 92 are suitably provided all the way around the wrist for complete coverage, but this is not a requirement, and another embodiment can use just a partial coverage, if just one side or portion of the wrist is to be cooled.

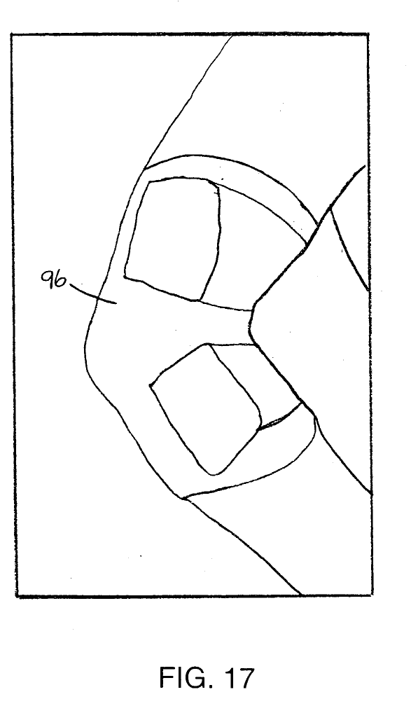

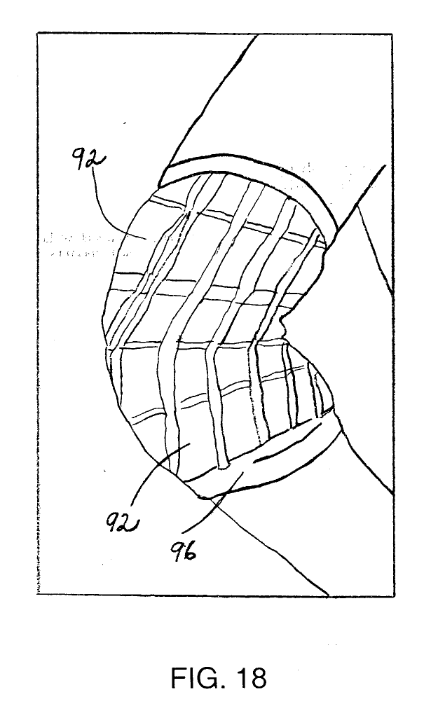

FIG. 17 is a photographic view of an elbow therapy wrap 96, and FIG. 18 is a cut away view thereof showing the placement of the cooling/heating elements 92.

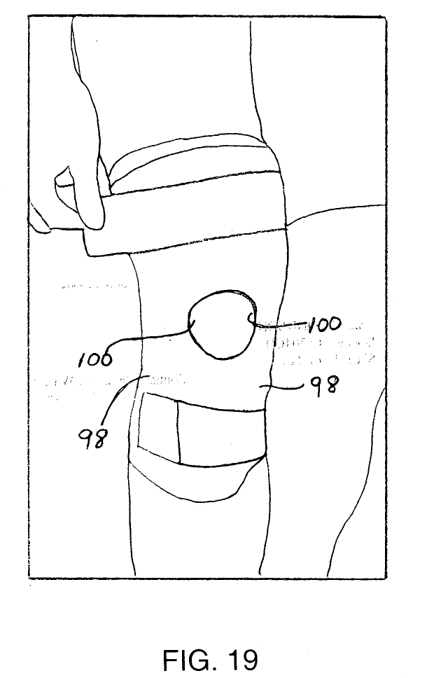

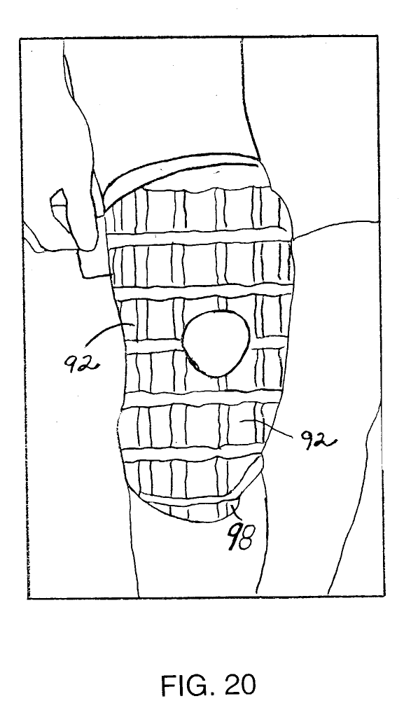

FIG. 19 is a photographic view of a knee therapy wrap 98, which suitably may include an opening 100 for alignment over the knee cap. FIG. 20 is a cut away view of the knee therapy wrap of FIG. 19, showing the placement of the cooling/heating elements 92 therein.

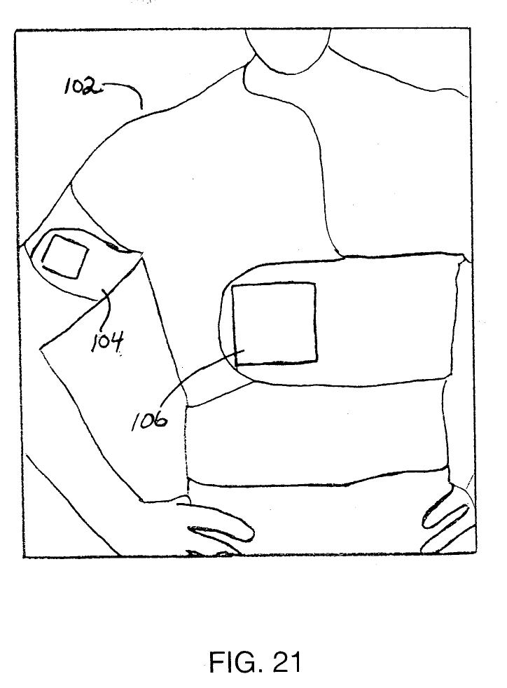

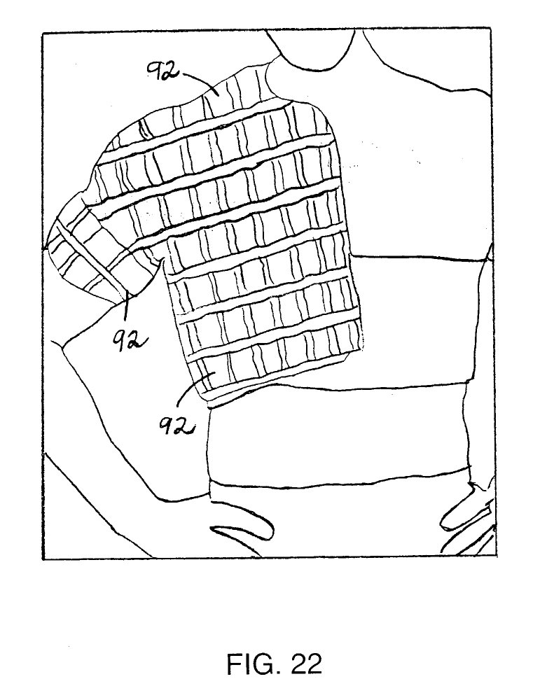

FIG. 21 is a photographic view of a shoulder therapy wrap 102, which is adapted to provide cooling to shoulder and chest areas (and upper back). The shoulder wrap includes both an arm fastener strap 104 and a chest strap portion 106, to enable secure attachment to the wearer. FIG. 22 is a cut away view of the shoulder therapy wrap of FIG. 21, illustrating the placement of the cooling/heating elements therein. Corresponding elements are suitably provided if desired on the back side (not illustrated).

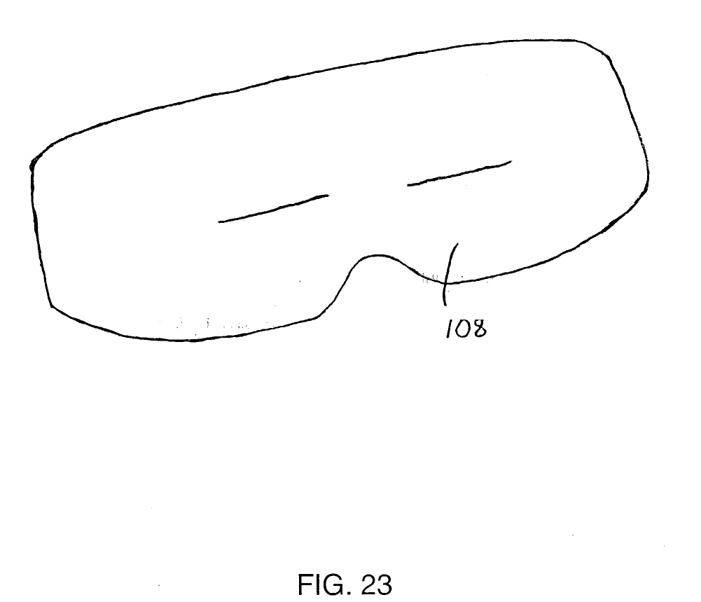

FIG. 23 is a photographic view of a face therapy mask 108, while FIG. 24 provides a cut away view of the face therapy mask of FIG. 23, illustrating the placement of the cooling/heating elements therein. A strap may be suitably provided (not shown) to enable the mask to be secured to the wearer's face. FIG. 28 is a drawing of the face therapy mask of FIG. 23, illustrating the placement of the cooling/heating elements 38 therein in phantom.

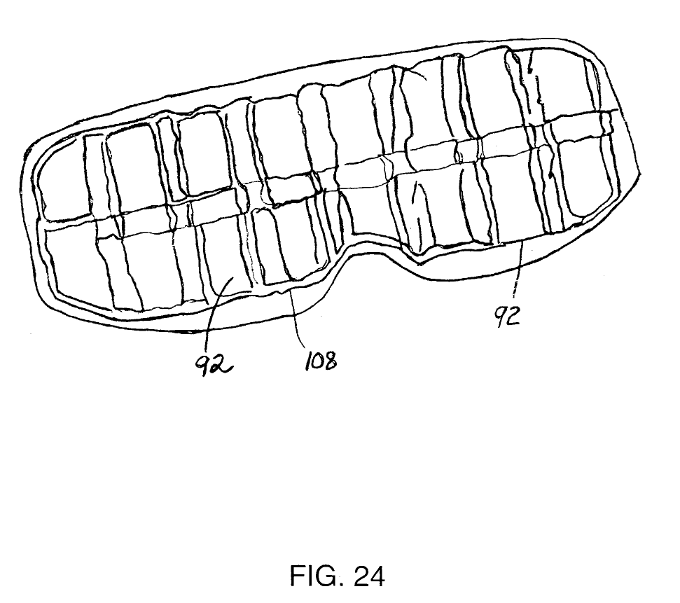

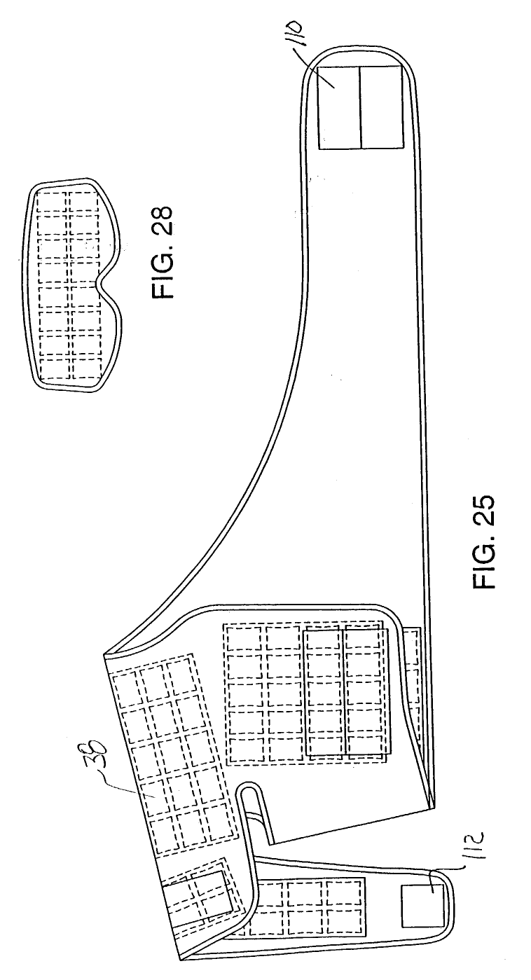

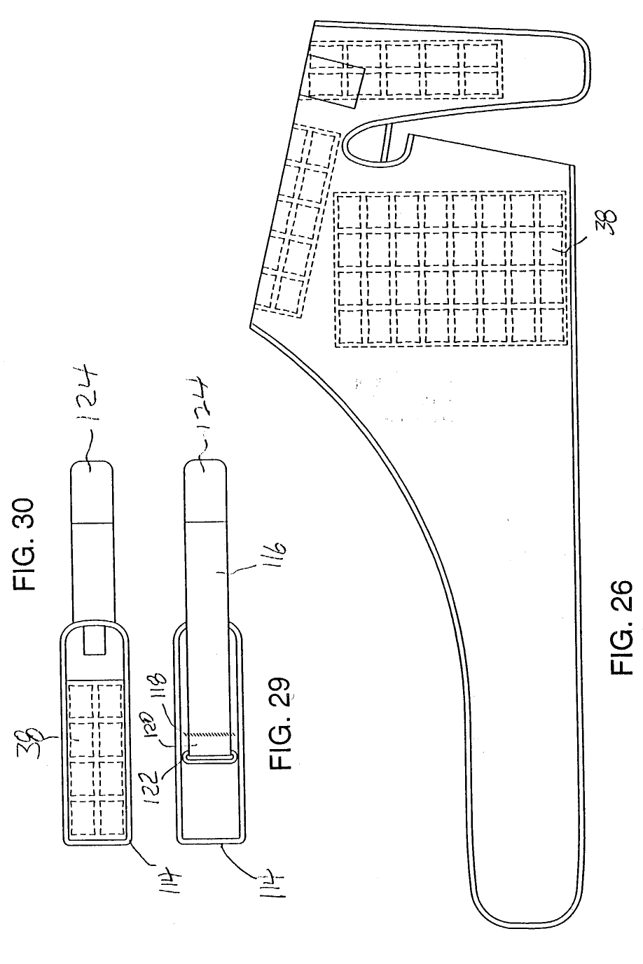

FIG. 25 is a front view drawing of a shoulder therapy wrap illustrating placement of cooling/heating elements 38 therein in phantom. The elements 38 are distributed in groups so as to position them to be near areas on the body that would be desirably heated or cooled when wearing the wrap. An elongate portion that suitably does not carry any elements 38 therein is provided, tapering to a narrower configuration, and carrying hook and loop fastener portions 110. This wrap has a folded over configuration on one side, and includes an arm hole portion, and is adapted to be worn by placing the wearer's arm through an arm opening, with the folded over region moved adjacent the side of the body. Then, an arm wrap portion having hook and loop fasteners 112 is wrapped around the arm, and the fasteners engage with either corresponding fastener portions on the outside of the wrap or to the surface of the wrap itself, to secure the wrap to the arm. Also, the portion carrying fasteners 110 is looped around the body and secured to attach the wrap to the user and hold it in place. FIG. 26 is a rear view of the wrap, illustrating that there are additional elements 38 provided at the back portion, so that when worn, as shown in FIG. 27, heat or cool therapy is applied to the shoulder, and to the upper quadrant of the wearer's body, and upper portion of the wearer's arm. The shoulder wrap covers shoulder, upper arm, pecks and lats portions of the body, and drives cold deep for soothing relief or to reduce swelling.

FIG. 29 is a front view of an alternate wrist therapy wrap, while FIG. 30 is a rear view thereof, and FIG. 31 is a view showing placement when worn by a user. This wrist wrap differs somewhat from the earlier described wrist wrap of FIG. 8. The wrap comprises an elongate body portion 114 which is flexible and which contains elements 38 therewithin. An elongate strap 116 is attached centrally of the portion 114, suitably by being sewn thereto as in the illustrated embodiment with an attachment or enforcement stitch 118. A loop portion 120 is also provided by the interaction of the strap 116 with the stitch 118, and a strap receiving hoop 120 is thereby loosely secured by the loop portion passing therethrough. An end portion of the strap 116 comprises hook and loop type fastener material.

Referring now to FIG. 31, to wear the wrist wrap, the user wraps the portion 114 around the wrist. Then, the strap 116 is wrapped around the outer portion of the wrist wrap, and the end of the strap is passed through the hoop 120. The strap is then pulled taut to tighten up the wrap onto the wrist, to whatever degree of tightness the wearer desires. The strap portion is then folded back, and by cooperation of the hook and loop fastener portion with the exposed face of the strap, is removably secured until the user wishes to remove the wrap. The wrist wrap can be used to reduce swelling and pain associated with carpal tunnel syndrome or wrist injury, for example.

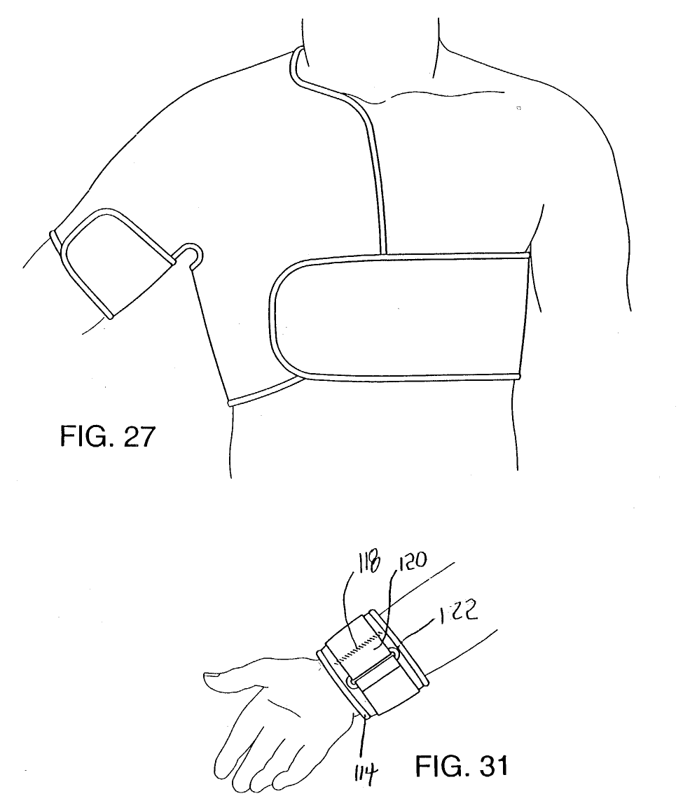

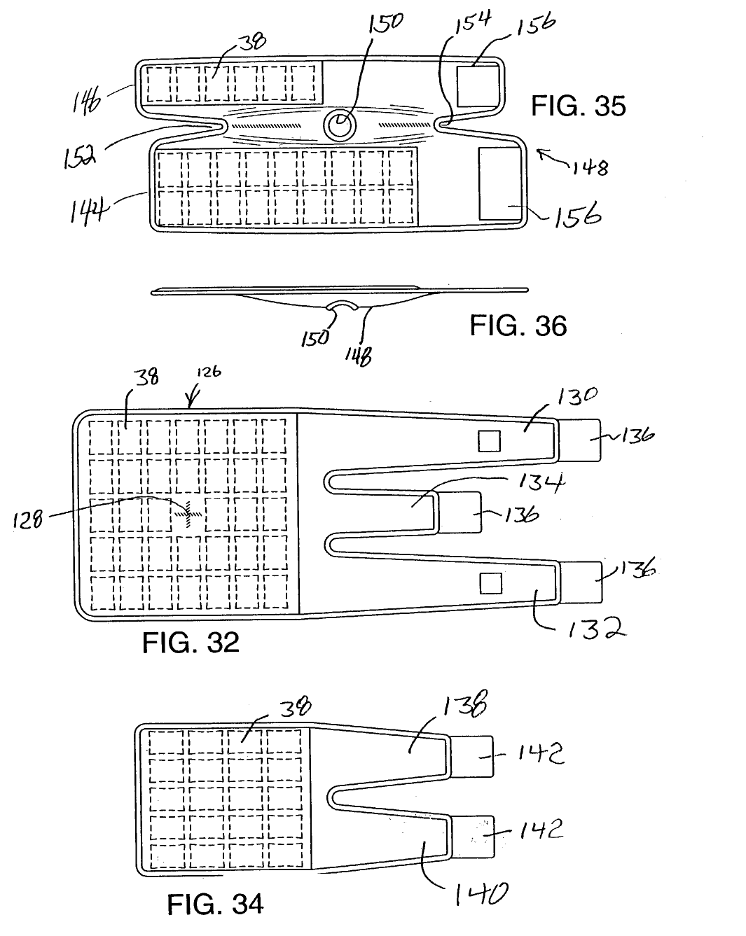

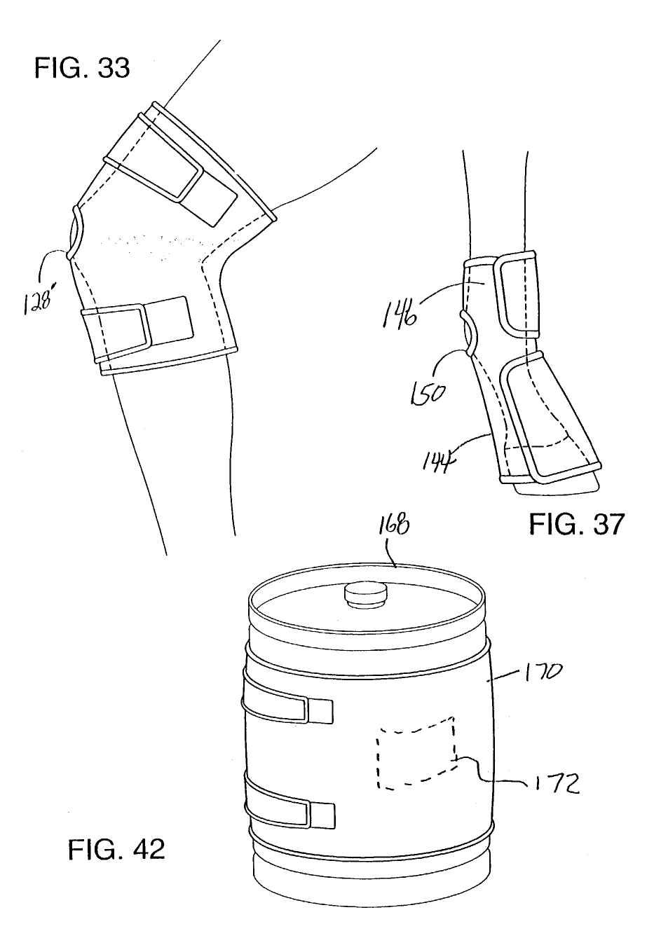

FIG. 32 is a view of a knee therapy wrap illustrating the placement of the cooling/heating elements therein in phantom. This embodiment of knee wrap has a substantially square shaped area 126 having an array of elements 38 therewithin. At the very center portion of the area 126, rather than have an element 38, a blank portion 128 is provided, with reinforcement stitching shown in the illustrated embodiment. An alternate embodiment (as in FIG. 33) can employ an opening rather than the blank portion. This portion 128 is to receive the knee cap thereagainst. In the wrap of FIG. 32, two longer strap members 130, 132 are provided at upper and lower portions (as viewed in the drawings), while a shorter, central strap 134 is optionally provided (strap 134 is not present in the embodiment of FIG. 33). Hook and loop type fastener portions 136 are provided at the distal ends of the various straps.

In use, the wearer will position the wrap with the portion 128 positioned over the knee cap, and will wrap the portion 126 around the leg. Then, straps 130, 132 (and 134 if present) are wrapped around the outside face of the wrap and are secured (by operation of the hook and loop fasteners, either with corresponding fastener members on the outer face of the wrap, or with the material of the wrap outer face itself). In FIG. 33, a view of an alternative wrap is given as worn on a knee. The wrap of this view does not employ the strap 134 and rather than having a “blank” or “no hole” portion 128, instead has an opening 128′ which allows the knee cap to be accommodated. The knee wrap also provides support even when unfrozen, giving dual use as a support and cold/heat therapy wrap.

FIG. 34 is a view of an elbow therapy wrap illustrating the placement of the cooling/heating elements therein in phantom. This particular wrap employs two straps 138, 140, with hook and loop fasteners 142 at the ends thereof. A cooling/heating element 38 carrying area of somewhat smaller size than the corresponding area of the knee wrap is provided. In this particular embodiment, plural elements 38 are contained within the wrap. The elbow wrap is attachable to an elbow in a manner similar to that of attaching the knee wrap to a user, but in this embodiment, no blank or opening portion is provided for the elbow bone region. However, it can be so provided, if desired. The elbow wrap prevents and controls tendonitis, bursitis and pain associated with arthritis, protects weak or painful joints during activity, and the adjustable straps reduce tension in wrist and finger extensors while gripping.

FIG. 35 is a view of a horse fetlock therapy wrap illustrating the placement of the cooling/heating elements therein in phantom. This wrap comprises a first band 144 of cooling elements and a second band 146 of cooling elements, positioned above the first band and spaced away therefrom by central fetlock receiving region 148. Approximately centrally of the fetlock receiving region, and opening 150 is provided to accommodate the fetlock. Cut in regions 152, 154 are provided on either side of the wrap to allow flexing of the upper and lower portions somewhat separately. Hook and loop type fastener portions 156 are provided at ends of the respective portions 144, 146. FIG. 36 is a side view of the wrap of FIG. 35, and illustrates how the central portion 148 bows outwardly with respect to the general plane of the wrap. FIG. 37 is a view of the horse fetlock therapy wrap when installed on a horse, for example. For use on a horse in this view, the narrower band 146 is positioned as the upper part of the wrap, while the lower part is the wider band 144, with opening 150 positioned at the fetlock. The horse therapy wrap prevents travel leg fatigue in horses without the use of messy ice and without requiring inserts.

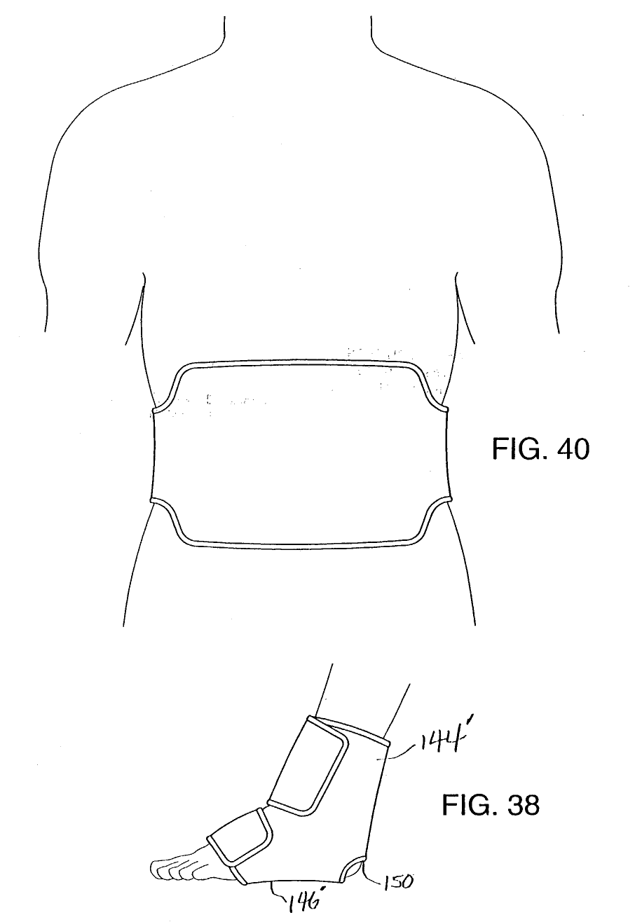

A similar configuration wrap is also employable (with size adjustment to accommodate the difference in size of a human user as contrasted with a horse) as an ankle wrap for humans. FIG. 38 is a view of an ankle therapy wrap as worn by a user. In this use, the narrower band of 146′ is employed as the lower end of the wrap, while the wider band 144′ becomes the upper wrap portion, the heel being accommodated by opening 150′. This wrap fits like an open toe, open heel sock. Its high spliced heel design provides added support and compression of Achilles tendons, and drives cold deep to help reduce swelling and prevent stiffness after injury. Extended coverage on top of foot reduces swelling in hard to reach areas.

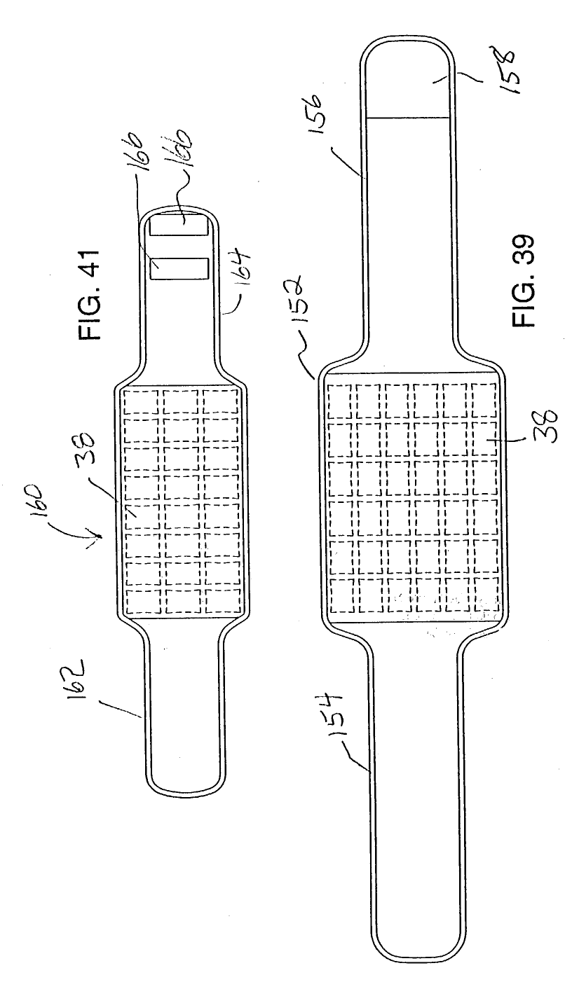

FIG. 39 is a view of a lower back therapy wrap illustrating the placement of the cooling/heating elements therein in phantom. A rectangular array of elements 38 is provided, centrally positioned with elongate left and right strap members 154, 156. A hook and loop fastener portion 158 is also provided.

FIG. 40 is a view of placement of the lower back therapy wrap of FIG. 39 when worn by a user. The body of the wrap provides breathable neoprene with refreezable inner elements, keeping the wearer dry and comfortable while providing soothing cold therapy. The construction helps retain body heat when elements 38 gels are unfrozen, while cold relieves pain, swelling and stiffness when elements 38 are frozen.

FIG. 41 is a view of a universal therapy wrap, illustrating the placement of the cooling/heating elements therein in phantom. This wrap is to accommodate a variety of non-specific uses, for example, hard to reach areas, face, eyes, jaw, for use when having migraine headaches, neck pain, hamstring, and calf strain. This particular embodiment employs plural elements 38, with an elongate rectangular array configuration 160, and strap portions 162, 164 and hook and loop fastener portions 166 on at least one distal strap end.

FIG. 42 is a view of a beer keg cooler wrap 170 as installed on a beer keg 168. A construction comprising a relatively large array of cooling elements 38 are contained within the wrap 170, as are elongate straps with hook and loop fattener elements thereon. In use, the wrap 170 is frozen, and is suitably wrapped around a keg of beer. The straps are further wrapped around the outside of the wrap and the hook and loop fasteners engage either the outer face of the wrap or corresponding hook and loop fastener members attached to the outer face of the wrap, to secure the wrap to the keg. In this manner, the keg is suitably kept cool. Further, the keg wrap is suitably adapted to have advertising 172 or other information thereon, such as the brand name of the beer in the keg, sports team information, or the like. The specific configuration of this wrap may be similar to that of the elbow wrap, with size and dimensional changes to accommodate the diameter, size and shape of the keg. A corresponding configuration of wrap may be used with other types of coolers holding beverages or the like.

The various wraps described hereinabove may find use in many sports and activities. For example, the elbow wrap, or the horse wrap, may suitably be used by bowlers, as an example.

In use, the wraps are wrapped around body parts to relieve pain and swelling. Two or more may be hooked together (via hook and loop fasteners) to enable attachment to the body, such as back, shoulder, upper leg or other larger body areas. The face mask may be used to provide relieve for migraines and headaches.

Cold therapy should be applied immediately after a minor injury when swelling is present. Cold offers relief over the course of the first 72 hours. Application of cold to the affected areas temporarily decreases blood flow to surrounding tissues, thereby minimizing localized pain and swelling. Cold therapy is suitably applied as follows, ON for 20 to 30 minutes, OFF for two hours. This ON/OFF cycle is repeated as desired, for example over a 3 day period.

Another part of cold therapy is compression, from wrapping the injured area to provide added support. The configuration of the cold therapy wraps of the invention enables them to provide both cold and compression. When the cold application is removed, another wrap that has not been cooled may be substituted for continued compression.

It will be noted that some or all of the above wraps suitably employ hook and loop type fasteners and straps, so as to enable attachment and adjustment. The fasteners may also suitably use only the hook type fastener portion, relying on the engagement of the hooks with the outer surface of the wrap for securing. In such case, the outer surface of the wrap acts as the loop portion of the hook and loop fastener pair.

By heating the wraps, they may also be used to provide heat therapy applications, if desired.

Accordingly, cold or heat therapy wraps are provided for humans or animals, which may be easily frozen or cooled, and when desired to be used, are taken from the freezer and installed on the person or animal receiving the cold therapy.

While plural embodiments of the present invention have been shown and described, it will be apparent to those skilled in the art that many changes and modifications may be made without departing from the invention in its broader aspects. The appended claims are therefore intended to cover all such changes and modifications as fall within the true spirit and scope of the invention.

What is claimed is:

1. A cooler, comprising:
a flexible side wall; and
at least one freezable element for providing cooling to an interior of said cooler.

2. A cooler according to claim 1, wherein said flexible side wall has insulating properties.

3. A cooler according to claim 1, wherein said flexible side wall comprises neoprene.

4. A cooler according to claim 1, wherein said flexible side wall comprises a neoprene outer layer and an inner liner layer.

5. A cooler according to claim 4, wherein said inner liner layer comprises a rubberized layer.

6. A cooler according to claim 4, wherein said inner liner layer comprises a stretchable rubberized layer.

7. A cooler according to claim 4, wherein said inner liner layer comprises a rubberized fabric layer.

8. A cooler according to claim 1, wherein said flexible side wall comprises a neoprene outer layer and an inner liner layer, wherein said at least one freezable element is positioned between said outer layer and said inner layer.

9. A cooler, comprising;
side walls adapted for folding to transform said cooler from an upright configuration to a substantially flat configuration; and
cooling elements positioned within at least one of said side walls.

10. A cold therapy wrap, comprising:
a flexible side wall; and
at least one freezable element for providing cooling to said wrap.

11. A cold therapy wrap according to claim 10, further comprising means for securing said cold therapy wrap to a user.

12. A cold therapy wrap according to claim 10, wherein said wrap is adapted for use on an animal and wherein said flexible side wall includes a fetlock receiving portion for receiving a fetlock joint portion of the animal therein.

13. A cold therapy wrap according to claim 10, further comprising hook and loop fastener portions for securing said wrap to a user.

14. A cold therapy wrap according to claim 10, wherein said at least one freezable element is a re-freezable element.

15. A therapy wrap, comprising:
a flexible side wall; and
at least one heatable/coolable/freezable element for providing heating or cooling to said wrap.

16. A therapy wrap according to claim 15, further comprising means for securing said therapy wrap to a user.

17. A therapy wrap according to claim 15, wherein said wrap is adapted for use on an animal and wherein said flexible side wall includes a fetlock receiving portion for receiving a fetlock joint portion of the animal therein.

18. A therapy wrap according to claim 15, further comprising hook and loop fastener portions for securing said wrap to a user.

19. A therapy wrap according to claim 15, wherein said wrap is adapted for use as a face therapy mask.

20. A therapy wrap according to claim 15, wherein said at least one heatable/coolable/freezable element is a re-heatable/re-coolable/re-freezable element.

21. A therapy wrap according to claim 15, wherein said wrap is adapted for use as a back therapy wrap.

22. A therapy wrap according to claim 15, wherein said wrap is adapted for use as a wrist or carpal tunnel therapy wrap.

23. A therapy wrap according to claim 15, wherein said wrap is adapted for use as an elbow therapy wrap.

24. A therapy wrap according to claim 15, wherein said wrap is adapted for use as a knee therapy wrap.

25. A therapy wrap according to claim 15, wherein said wrap is adapted for use as a shoulder therapy wrap.

26. A cooler device for use with a beverage containing vessel, comprising:
a flexible side wall; and
at least one coolable/freezable element for providing or cooling to said flexible side wall.

27. A cooler device according to claim 26, wherein said beverage containing vessel is a keg.

28. A cooler device according to claim 26, further comprising at least one attachment member for securing said flexible sidewall to the vessel.
